(19) United States
(12) Patent Application Publication
(10) Pub. No.: US 2017/0281607 A1
(43) Pub. Date: Oct. 5, 2017

(54) PHARMACEUTICAL COMPOSITIONS AND METHODS FOR THE TREATMENT OF DIABETES

(71) Applicant: University of Limerick, Limerick (IE)

(72) Inventor: Mark Davies, Limerick (IE)

(21) Appl. No.: 15/474,430

(22) Filed: Mar. 30, 2017

Related U.S. Application Data
(60) Provisional application No. 62/377,920, filed on Aug. 22, 2016.
(60) Provisional application No. 62/376,485, filed on Aug. 18, 2016.
(60) Provisional application No. 62/316,931, filed on Apr. 1, 2016.

(51) Int. Cl. A61K 31/437; A61K 45/06

(52) U.S. Cl. CPC A61K 31/437; A61K 47/48038; A61K 45/06

(57) ABSTRACT

This invention relates to compositions and methods for treating type I diabetes. Compositions of the invention include active compounds such as harmine, INDY, or derivatives thereof to induce reproduction of beta cells. These active compounds are provided in amounts that have poor clinical efficacy on their own, but when administered at a sub-threshold dose in combination with compounds such as insulin, which helps to maintain steady levels of insulin in the body; metformin, which decreases glucose levels; and repaglinide, which increases insulin production, allow for the slow and steady increase of beta cells.

### RELATED APPLICATIONS

The present application claims the benefit of and priority to U.S. Provisional Application No. 62/316,931, filed Apr. 1, 2016; U.S. Provisional Application No. 62/376,485, filed Aug. 18, 2016; and U.S. Provisional Application No. 62/377,920, filed Aug. 22, 2016, the contents of each of which are incorporated by reference herein in their entirety.

### FIELD

This invention relates to compositions and methods for the treatment of diabetes.

### BACKGROUND

Diabetes mellitus affects hundreds of millions of people worldwide and can be fatal. There are two main types of diabetes mellitus, Type I and Type II. Type 1 diabetes results from the failure of the pancreas to produce sufficient insulin due to the loss of insulin-producing beta cells in the Islets of Langerhans. The cause of Type I diabetes is not known, although a genetic link has been proposed; and there are multiple genes thought to influence the risk of diabetes. Type II diabetes results from a failure of cells to respond to insulin appropriately. The cause of Type II diabetes is usually attributed to excessive body weight and lack of exercise, in addition to genetic factors.

Insulin is released into the blood by the beta cells ((β-cells) in response to increasing levels of blood glucose, typically after eating. Insulin is used by a majority of the body's cells to absorb glucose from the blood for many essential processes. Lower glucose levels result in decreased insulin release from the beta cells. If an insufficient amount of insulin is available, if cells respond poorly to the effects of insulin (insulin insensitivity or insulin resistance), or if the insulin itself is defective, then glucose will not be absorbed properly by cells. The resulting high levels of blood glucose can lead to many issues, such as poor protein synthesis and other metabolic disorders.

Conventional treatment for Type I diabetes is primarily in the form of insulin injections and pancreatic transplants. Type II diabetes is usually treated with oral medications, such as metformin, and/or insulin injections. In addition to metformin, several other drugs have been used to treat Type II diabetes, such as agents that increase insulin release (e.g., repaglinide), agents that decrease absorption of sugar from the intestines, and agents that make the body more sensitive to insulin. Repaglinide (Prandin®) belongs to a class of medications, known as meglitinides, that promotes insulin release from beta cells. Metformin and repaglinide are limited to use in Type II diabetes and, in fact, are contraindicated for treatment with Type I diabetes.

Studies have shown that harmine (C₁₃H₁₂N₂O), may induce beta cell proliferation, increase islet mass, and improve glycemic control in Type I diabetics. Harmine is a beta carboline compound that inhibits DRK1A receptors and is a member of the dual-specificity tyrosine-regulated kinase (DYRK) family. Harmine also reversibly inhibits monoamine oxidase A. INDY, a DYRK1A inhibitor (but not a MOA inhibitor) has also been found to activate proliferation in both rat and human beta cells. Both harmine and INDY were also shown to induce phosphorylation of histone, a marker of cell cycle transition, which ultimately affects insulin regulation.

Use of harmine is restricted by regulatory authorities due to its psychotoxic effects at therapeutic doses. Furthermore, treatment with harmine (or INDY) alone at levels sufficiently low to reduce toxicity to an acceptable level likely would not provide clinically successful treatment of diabetes. Thus, although harmine and INDY have been shown to be promising in promotion of beta cell growth, there exists a need to develop a composition and method for administering a beta cell replicator, such as harmine or a derivative thereof, that does not result in toxicity and is useful in continued treatment of diabetes.

### SUMMARY

The invention provides compositions and methods for the treatment of diabetes through the induction of beta cell production and regulation of glucose and insulin. Compositions and methods of the invention combine a plurality of active compounds, some of which, as previously used, would either be ineffective or would result in toxicity, but that in practice of the invention produce a low-toxicity therapeutic benefit.

In certain embodiments, a Beta cell promoter is provided in combination with insulin and/or insulin-support drugs in order to stimulate healthy Beta cell production and function. For example, various combinations of harmine, INDY, insulin, metformin, repaglinide and/or derivatives thereof are provided to induce reproduction of healthy beta cells. In a preferred embodiment harmine or INDY is provided in combination with insulin, as described below. In another embodiment, harmine or INDY is provided in combination with either metformin or repaglinide or both metformin and repaglinide as described below. In yet another embodiment, harmine or INDY is provided in combination with insulin and with either metformin or repaglinide or both.

Harmine (or INDY) is provided in an amount that has a poor clinical efficacy on its own. Insulin is provided to help maintain steady levels of insulin in the body. Metformin is provided to decrease glucose levels, while repaglinide is provided to increase insulin production. In this way, a patient benefits from the insulin regulation provided by insulin, metformin and/or repaglinide, while allowing for the slow and steady increase of beta cells induced by a sub-threshold amount of harmine (or INDY). In certain aspects active compounds are provided in amounts sufficient to induce replication of beta cells at a faster rate than the cells are being depleted. In other embodiments, harmine or INDY is combined with one or all of insulin, metformin and repaglinide in order to achieve the clinical benefit. As discussed below, various combinations of the compounds work as long as harmine or INDY is provided in an amount that would not provide a benefit if administered alone to a patient with no detectable circulating level of the drug. Drug cocktails as provided herein can be titrated or calibrated to allow a gradual reduction in the requirement for insulin therapy.

In an embodiment of the invention, a pharmaceutical composition is provided that includes harmine in an amount insufficient to stimulate pancreatic beta cell production if the same amount were given to a patient having an undetectable level of circulating harmine. The composition can also include one or all of insulin, repaglinide and metformin; and a pharmaceutically-acceptable excipient, diluent, or carrier. All active compounds of the composition can be used in a pharmaceutically-acceptable salt form. It is also to be understood that derivatives of harmine, such as prodrugs, or other active compounds known to induce replication of beta cells, such as INDY or proINDY, can be provided in place of harmine.

In one aspect, a derivative of harmine is provided, wherein harmine is conjugated to a blocking group. The blocking group may be any acceptable group including but not limited to hydroxyls, amines, carboxyls and carbonyls. In a preferred embodiment, an acetate-conjugated harmine comprises the following formula:

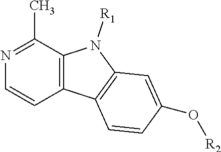

wherein R₁ is C₂H₃O and R₂ is H; or R₁ is H and R₂ is C₂H₃O.

In certain aspects, insulin is present in the composition at an amount sufficient to establish a basal level of circulating insulin. In certain aspects, repaglinide and/or metformin is present in the composition in an amount about at or below a threshold dose necessary to stimulate insulin production when administered alone. Repaglinide and/or metformin can also be present in the composition in a pro-drug form.

In other aspects, pharmaceutical compositions of the invention include harmine in an amount that would be insufficient to stimulate pancreatic beta cell production in a single administration to a patient when the concentration of harmine in the patient is zero. Compositions of the invention can be provided in an injectable dosage form.

### BRIEF DESCRIPTION OF THE DRAWINGS

FIG. 1 shows process for preparing a controlled release dosage form using nanoparticle technology. FIG. 2 illustrates the formation of a nisome for controlled release of a drug. FIG. 3 illustrates a microencapsulation controlled release dosage form. FIG. 4 illustrates the formation of a liposome for controlled release of a drug. FIG. 5 illustrates a process for osmotically controlling release of a drug. FIG. 6 illustrates a process for controlled release of a drug through the use of an ion exchange resin.

### DETAILED DESCRIPTION

Pharmaceutical compositions of the invention include a combination of active compounds that are effective in the treatment of diabetes. Compounds are provided that induce beta cell reproduction, such as harmine, INDY, and derivatives thereof. Insulin or insulin analogs are also provided, either separately or as part of the same composition. Compounds that increase insulin production and decrease glucose production, such as repaglinide and metformin, are also provided. The combination of these compounds results in a synergistic effect in the treatment of diabetes, including Type I diabetes, through the slow replication of beta cells and regulation of insulin.

Harmine is 7-Methoxy-1-methyl-9H-pyrido[3,4-b]indole and is a fluorescent harmala alkaloid belonging to the beta-carboline family of compounds having the following structural formula:

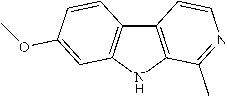 [SMILES: COc1ccc2c(c1)[nH]c1c(C)nccc12]

Harmine is a dual-specificity tyrosine-regulated kinase (DYRK) family, monoamine oxidase-A (MOA-A), and cdc-like kinases (CLK) inhibitor, that has recently been shown to induce beta cell replication. Harmine has been shown to inhibit the telomerase activity of MCF-7 cells by down-regulating hTERT mRNA expression accompanied by an accelerated senescent phenotype.

In one embodiment of the invention, harmine derivatives are provided. The derivatives can be prodrugs of harmine. Prodrugs are compounds that, after administration, are metabolized into a pharmacologically active drug. Prodrugs are often used instead of the pharmacologically active drug to improve how a medicine is absorbed, distributed, metabolized, and excreted. In many cases the drug itself is poorly absorbed from the gastrointestinal track, so the use of a prodrug can improve bioavailability. Additionally, a prodrug can improve the selectivity of a drug, such that interaction with unintended targets is minimized or eliminated.

Prodrugs can be formed by conjugating the drug to a protecting group. Examples of suitable protecting groups include, but are not limited to, the following: carbobenzyloxy (Cbz) group, p-methoxybenzyl carbonyl (Moz or MeOZ) group, tert-butyloxycarbonyl (BOC) group, 9-fluorenylmethyloxycarbonyl (FMOC) group, acetyl (Ac) group, benzoyl (Bz) group, benzyl (Bn) group, carbamate group, p-methoxybenzyl (PMB), 3,4-dimethoxybenzyl (DMPM), tosyl (Ts) group, and other sulfonamides (Nosyl & Nps) groups.

In one embodiment, harmine is conjugated to an acetyl group. In one aspect, the prodrug is a compound selected from the following formula:

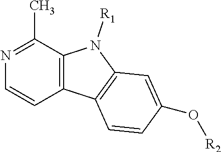

wherein R₁ is C₂H₃O and R₂ is H or R is Hand R₂ is C₂H₃O.

INDY is a DYRK1 inhibitor having the chemical name (1Z)-1-(3-ethyl-5-hydroxy-1,3-benzothiazol-2-ylidene)propan-2-one and the following structural formula:

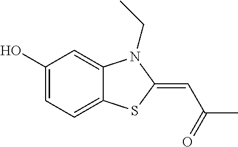 [SMILES: CCN1/C(=C/C(C)=O)Sc2ccc(O)cc21]

INDY binds at the ATP-binding cleft of the DYRK1 enzyme, reverses aberrant tau-phosphorylation and rescues repressed calcineurin/NFAT signaling. As with harmine, INDY has recently been shown to induce beta cell replication. INDY is often used in its prodrug form ProINDY.

ProINDY is the prodrug of INDY having the chemical name (1Z)-1-(5-Acetyloxy3-ethyl-2(3H)-benzothiazolylidene)-2-propanone and the following structural formula:

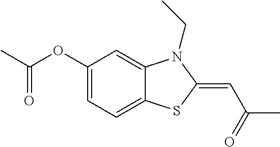 [SMILES: CCN1/C(=C/C(C)=O)Sc2ccc(OC(C)=O)cc21]

Insulin is a peptide hormone normally produced by Beta cells in the pancreas. Insulin can include human or non-human, recombinant, purified or synthetic insulin or insulin analogues. Biosynthetic human insulin can be produced for clinical use by recombinant DNA technology. Insulin analogs are also available for clinical use. An insulin analog is insulin altered through, for example, genetic engineering of the underlying DNA or chemical modification, such as by acetylation. Examples of insulin analogs include insulin lispro, insulin glargine, insulin aspart, insulin glulisine, insulin detemir. Some analogs are fast acting (aspart, lispro, and glulisine) and some are longer acting (glargine and detemir). Some clinical insulin products are a combination of both short and longer acting insulin/insulin analogs. Currently, insulin and insulin analogs are not available in oral dosage forms due to its breakdown upon being introduced to the gastrointestinal tract. Instead, insulin and/or its analogs are typically administered by injection.

Repaglinide (brand name PRANDIN®) has a chemical name S-(+)-2-Ethoxy-4-(2((3-methyl-1-(2-(1-piperidinyl)phenyl)butyl)amino)2-oxoethyl) benzoic acid and is used to treat diabetes. See e.g., U.S. Patent Publication No. 2009/0209587, U.S. Pat. No. 5,312,924 and U.S. Pat. No. RE37035, the contents of which are incorporated herein by reference. It belongs to the meglitinide class of insulin secretagogues, compounds that stimulate insulin release from the pancreas. Repaglinide has the following structural formula:

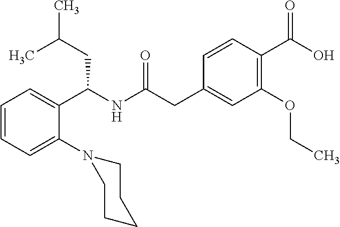 [SMILES: CCOc1cc(CC(=O)N[C@@H](CC(C)C)c2ccccc2N2CCCCC2)ccc1C(=O)O]

Repaglinide is indicated as an adjunct to diet and exercise to lower the blood glucose in patients with type 2 diabetes mellitus (non-insulin dependent diabetes mellitus, or NIDDM). In one embodiment, repaglinide can be provided in compositions of the invention in a prodrug form. Processes for preparing repaglinide are known in the art. See e.g., US Patent Application No. 2009/0209587, incorporated by reference in its entirety herein.

Metformin (brand name GLUCOPHAGE®, among others) has the chemical name N,N-Dimethylimidodicarbonimidic diamide and the following structural formula:

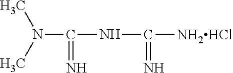

Metformin decreases hyperglycemia primarily by suppressing glucose production by the liver (hepatic gluconeogenesis). It is used for treatment of Type II diabetes.

Methods for preparing metformin and pharmaceutically acceptable salts thereof are known in the art and are described, for example, in U.S. Pat. Nos. 3,174,901 and 6,031,004, the contents of which are incorporated by reference herein in their entirety. In one embodiment, metformin can be provided in compositions of the invention in a prodrug form.

Compounds of the invention can be administered as one of their pharmaceutically acceptable salts. Examples of pharmaceutically acceptable salts include, but are not limited to, the hydrochloride, acetate, benzoate, citrate, fumarate, embonate, chlorophenoxyacetate, glycolate, palmoate, aspartate, methanesulphonate, maleate, parachlorophenoxyisobutyrate, formate, lactate, succinate, sulphate, tartrate, cyclohexanecarboxylate, hexanoate, octonoate, decanoate, hexadecanoate, octodecanoate, benzenesulphonate, trimethoxybenzoate, paratoluenesulphonate, adamantanecarboxylate, glycoxylate, glutamate, pyrrolidonecarboxylate, naphthalenesulphonate, 1-glucosephosphate, nitrate, sulphite, dithionate or phosphate. Preferred salts include the hydrochloride, fumarate, embonate and chlorophenoxyacetate salts. The pharmaceutically acceptable salts of the compounds can be obtained by the action of the compound on the corresponding acid.

Compositions of the invention can include a pharmaceutically acceptable amount of one or more of the following active compounds: harmine, a derivative of harmine, INDY, ProINDY, insulin, repaglinide, metformin, and combinations thereof. In one embodiment, the composition includes harmine or a prodrug of harmine and insulin. In another embodiment, the composition comprises harmine, insulin, repaglinide and/or metformin. In yet another embodiment, the composition includes INDY or proINDY and insulin. In another embodiment, the composition comprises INDY (or proINDY), insulin, repaglinide and/or metformin. In one embodiment, the composition includes harmine, repaglinide and metformin. In another embodiment, a composition of the invention comprises a prodrug of harmine, repaglinide, and/or metformin. In yet another embodiment, the composition includes INDY, repaglinide and/or metformin. Another embodiment includes proINDY, repaglinide and metformin. In each case, the amount of harmine or INDY (or of their prodrugs) is less than an amount that would stimulate beta cell production in a patient having no detectable levels of the drug prior to administration. In one aspect of the invention, the composition is provided in a single dosage form. To be clear, compositions of the invention may be administered in multiple doses over time or in a controlled-release formulation and patients may, prior to second and subsequent doses, have detectable levels of harmine or INDY. The invention contemplates that the amount of harmine or INDY in the claimed compositions is less than an amount that would stimulate beta cell production IF administered to a patient with no detectable level of the drug. In another aspect of the invention, the compounds are provided separately as part of a kit, each compound in the kit to be administered separately yet simultaneously.

The effective dosage of each active compound can readily be determined by a skilled person, with regard to typical factors such as the age, weight, gender, blood sugar levels, food intake, and clinical history of the patient. A typical dosage of each compound, except for insulin, could be, for example, 0.01-1,000 mg/kg, preferably 0.1-500 mg/kg per day, for example. A typical dosage of insulin could be, for example, between about 20-90 pmol/l. The compounds can be administered one, two, three, four, five, six, ten or more (especially with respect to the insulin) times per day, every other day, every few days, once a week, once every two weeks, or once a month, or a limited number of times, such as just once, twice or three or more times. It may be advisable to start a patient on a low dose combination and work up gradually to a high dose combination.

In accordance with certain aspects, repaglinide can be provided in compositions of the invention in an amount equal to or less than about 16 mg/day or a dosage of about 0.5 to about 4 mg orally. In one embodiment, repaglinide is provided in compositions of the invention in an amount between about 0.1 mg and about 10 mg, such as at about 0.1 mg, 0.25 mg, 0.5 mg, 1 mg, 2 mg, 3 mg, 5 mg, 7 mg, 10 mg, and any amount in between. In one aspect, repaglinide is present in the dosage form at about or below a threshold dose necessary to stimulate insulin production when administered alone.

In accordance with certain aspects, metformin can be provided in compositions of the invention in an amount equal to or less than about 2550 mg per day. In one embodiment, metformin is provided in compositions of the invention in an amount between about 50 mg and about 750 mg, such as at about 50 mg, 100 mg, 200 mg, 250 mg, 300 mg, 400 mg, 500 mg, 600 mg, 750 mg, and any amount in between. In one embodiment, metformin is provided in compositions of the invention in an amount between about 100 mg/day and about 3000 mg/day, such as at about 850 mg/day.

In accordance with certain aspects, harmine or any derivative thereof can be provided in compositions of the invention in an amount equal to or less than about 30 mg/kg. In one embodiment, harmine or its prodrug can be provided in compositions of the invention in an amount between 0.1 mg/kg and about 10 mg/kg, such as at about 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, or 9 mg/kg. In one aspect, harmine or any derivative thereof is present in an amount insufficient to stimulate pancreatic beta cell production if said amount were given to a patient having an undetectable level of circulating harmine, or in an amount that would be insufficient to stimulate pancreatic beta cell production in a single administration to a patient when the concentration of harmine in the patient is zero.

In accordance with certain aspects, INDY or proINDY can be provided in compositions of the invention in an amount equal to or less than about 10 mg/kg. In one embodiment, metformin or replaglinide is provided in compositions of the invention in an amount between about 100 mg/day and about 3000 mg/day, such as at about 850 mg/day, and any amount in between. In one aspect, INDY or proINDY can be present in an amount insufficient to stimulate pancreatic beta cell production if said amount were given to a patient having an undetectable level of circulating INDY, or in an amount that would be insufficient to stimulate pancreatic beta cell production in a single administration to a patient when the concentration of INDY in the patient is zero.

Typical Type I diabetes inject insulin once per day, usually by subcutaneous injection. According to the invention, harmine is introduced at arrange from about 0.1 mg/kg to about 10 mg/kg, and preferably at about 5.05 mg/kg. Insulin is added at a dosage range of about 50-90 pmol/l (the typical physiological range), with a minimum of about 50 pmol/l. Insulin can be formulated alone or in pharmaceutical combination with harmine. In any event a preferred harmine-to-insulin ratio is about 5.05 mg/kg +/−4.9 mg/kg harmine to about 50-90 pmol/l of insulin. As the effects of harmine induce beta cell proliferation, the amount if insulin can be reduced by as much as 40 pmol/l.

The active compounds of the invention can be present at various ratios. For example, harmine and insulin can be present in a ratio of 0.1-10 mg/kg: 20-90 pmol/l, respectively.

In one aspect wherein repaglinide and/or metformin are also provided in addition to insulin, harmine, repaglinide and metformin can be present in a ratio of about 1:1:1, 100:1:100, 1:100:100, 100:100:1. In addition, the repaglinide/metformin ratio can be kept constant and harmine can be varied (e.g., 1:250:1). In carrying out the methods of the present invention, the pharmaceutical compositions of the invention can be administered to any mammalian species, such as monkeys, dogs, cats, rats, humans, etc.

Suitable routes of administration include oral, buccal, topical (including trans-dermal), subcutaneous, intradermal, intramuscular, intravenous, nasal, pulmonary, and with or on an implantable medical device (e.g., stent or drug-eluting stent or balloon equivalents).

Examples of suitable forms of oral pharmaceutical compositions include, but are not limited to, tablets, troches, lozenges, fast-melts, aqueous or oily suspensions, liquids, dispersible powders or granules, emulsions, hard or soft capsules, or syrups or elixirs.

Compositions may be prepared according to any method known in the art for the manufacture of pharmaceutical compositions. In addition to the active compounds discussed above, compositions of the invention can contain one or more pharmaceutically acceptable excipients and adjuvants.

Pharmaceutically acceptable excipients for us in solid oral compositions can include but are not limited to any one or more of diluents, fillers, binders, glidants, lubricants, basifying agents, surfactants, colorants, flavors, and solvents.

Suitable fillers or diluents include but are not limited to cellulose derivatives, such as microcrystalline cellulose or wood cellulose (including microcrystalline cellulose 302), lactose, lactose anhydrous, sucrose, starch, pregelatinized starch, dextrose, mannitol (including mannitol Pearlitol SD 200), fructose, xylitol, sorbitol, corn starch, modified corn starch, potato starch, rice starch, wheat starch, inorganic salts such as calcium carbonate, calcium phosphate, dicalcium phosphate, tribasic calcium phosphate, calcium sulfate, and sodium carbonate, dextrin/dextrates, maltodextrin, compressible sugars, and other known bulking agents or fillers, and/or mixtures of two or more thereof. Several types of microcrystalline cellulose are suitable for use in the formulations described herein, for example, microcrystalline cellulose selected from the group consisting of Avicel® types: PH101, PH102, PH103, PH105, PH 112, PH113, PH200, PH301, and other types of microcrystalline cellulose, such as silicified microcrystalline cellulose. Several types of lactose are suitable for use in the formulations described herein, for example, lactose selected from the group consisting of anhydrous lactose, lactose monohydrate, lactose fast flow, directly compressible anhydrous lactose, and modified lactose monohydrate.

Suitable binders include, but are not limited to, methyl cellulose, carboxymethyl cellulose (including sodium carboxymethyl cellulose), ethyl cellulose, hydroxypropyl cellulose (including HPC-SSL, HPC-SL, HPC-L, HPC-EXF, HPC-ELF, etc.), corn starch, pregelatinized starch, modified corn starch, dextrin, maltodextrin, polyvinyl pyrrolidone (PVP), povidones (PVP-K25, PVP-K29, PVP-K30, PVP-K90), Plasdone™ S 630 (copovidone), hydroxypropyl methylcellulose (HPMC) (including hydroxypropyl methylcellulose 2208), lactose, gum acacia, gum arabic, gelatin, agar, ethyl cellulose, cellulose acetate, tragacanth, alginic acid, sodium alginate, pullulan, polymethacrylate, as well as a wax binder such as carnauba wax, paraffin, spermaceti, polyethylenes or microcrystalline wax, as well as other conventional binding agents and/or mixtures of two or more thereof. Preferred binders of the present invention are hydroxypropyl cellulose SSL, hydroxypropyl cellulose SL, hydroxypropyl cellulose ELF, polyvinyl alcohol-polyethylene glycol, and polyvinyl pyrrolidone. The most preferred binder is hydroxypropyl cellulose SSL.

Suitable disintegrants include, but are not limited to, croscarmellose sodium, crospovidone (crospovidone XL-10, Kollidon CL®, Polyplasdone XL®, Kollidon Polyplasdone XL-10®, and Polyplasdone INF-10®), colloidal silicon dioxide, starch, potato starch, pregelatinized starch, corn starch, sodium carboxyl methylcellulose, sodium starch glycolate, microcrystalline cellulose, low substituted hydroxypropyl cellulose LH21, polyvinyl pyrrolidone cross linked, guar gum, magnesium aluminum silicate, potassium polacrilin, cellulose powder, sodium alginate, and other known disintegrants. Resins may also be used as disintegrants. Nonlimiting examples of useful resins include Amberlite® IR-120 Plus (H), Amberlite® IR-120 Plus, Amberlite® IRP-69, Amberlite® 15, Amberlite® 1200 (H), Amberlite® IRP-88, Amberlite® IRP-64, Dowex®1×2-100, 200, 400; 1×4-50, 100, 200, 400; 1×8-50, 100, 200, 400, and Duolite C-26.

Examples of glidants and/or anti-adherents suitable for use herein include but are not limited to, silicon dioxide, colloidal silicon dioxide, magnesium silicate, magnesium trisilicate, talc, and other forms of silicon dioxide, such as aggregated silicates and hydrated silica, and mixtures thereof.

Various lubricants that can be used include but are not limited to stearic acid and stearic acid derivatives such as magnesium stearate, calcium stearate, zinc stearate, sucrose esters of fatty acid, polyethylene glycol, talc, sodium stearyl fumarate, Amberlite IRP88, castor oils, waxes, palmitic acid, sodium laurel sulfate, glyceryl monostearate, glyceryl palmitostearate, sodium benzoate, myristic acid and hydrogenated vegetable oils and fats, as well as other known lubricants, and/or mixtures of two or more thereof.

Suitable basifying agents include but are not limited to, for example, sodium hydroxide, potassium hydroxide, calcium hydroxide, sodium carbonate, ammonia, diethanolamine, meglumine, lysine, arginine, ethanolamine, piperazine, trometamol and triethanolamine, ammonia, etc.

Suitable surfactants include but are not limited to sodium lauryl sulfate, dioctylsulfosuccinate, gelatin, casein, lecithin (phosphatides), dextran, gum acacia, cholesterol, tragacanth, hydroxypropyl methylcelluloses, hydroxypropylcelluloses, polyvinylpyrrolidones, polyoxyethylene alkyl ethers (e.g., macrogol ethers such as cetomacrogol 1000), polyoxyethylene castor oil derivatives, polyoxyethylene sorbitan fatty acid esters (such as the commercially available Tween™ products, e.g., Tween 20 and Tween 800 (ICI Speciality Chemicals)), polyethylene glycols (e.g., Carbowax 3550 and 934 (Union Carbide)), polyoxyethylene stearates, carboxymethylcellulose calcium, carboxymethyl cellulose sodium, methylcelluloses, hydroxyethylcelluloses, hydroxypropyl methylcellulose phthalates, magnesium aluminium silicate, triethanolamine, polyvinyl alcohols (PVA), poloxamers (e.g., Pluronic™ products F68 and F108Q, which are block copolymers of ethylene oxide and propylene oxide); poloxamines (e.g., Tetronic™908, also known as poloxamine 908, which is a tetrafunctional block copolymer derived from sequential addition of propylene oxide and ethylene oxide to ethylenediamine (BASF Wyandotte Corporation, Parsippany, N.J.)), Tetronic™15080 (T-1508) (BASF Wyandotte Corporation), PEG-derivatized phospholipids, PEG-derivatized cholesterols, PEG-derivatized cholesterol derivatives, PEG-derivatized vitamin A, PEG-derivatized vitamin E, lysozyme, random copolymers of vinylpyrrolidone and vinyl acetate, and the like.

Various useful colorants include but are not limited to Food Yellow No. 5, Food Red No. 2, Food Blue No. 2, and the like, and pigments such as iron oxides.

Flavoring agents, which can be used in the present invention, include but are not limited to flavors having a natural or synthetic or semi synthetic origin like menthol, fruit flavors, citrus oils, peppermint oil, spearmint oil, and oil of wintergreen (methyl salicylate).

Various film forming agents that can be used include but are not limited to cellulose derivatives such as soluble alkyl- or hydroalkyl-cellulose derivatives such as methyl celluloses, hydroxymethyl celluloses, hydroxyethyl celluloses, hydroxypropyl celluloses, hydroxymethyethyl celluloses, hydroxypropyl methylcelluloses, sodium carboxymethyl celluloses, etc., acidic cellulose derivatives such as cellulose acetate phthalates, cellulose acetate trimellitates and methylhydroxypropylcellulose phthalates, polyvinyl acetate phthalates, etc.; insoluble cellulose derivatives such as ethyl celluloses and the like; dextrins, starches and starch derivatives, polymers based on carbohydrates and derivatives thereof, natural gums such as gum Arabic, xanthans, alginates, polyacrylic acids, polyvinyl alcohols, polyvinyl acetates, polyvinylpyrrolidones, polymethacrylates such as derivatives thereof (Eudragit™), chitosan and derivatives thereof, shellac and derivatives thereof, and waxes and fat substances. The film-forming agent can be present in an amount from about 10% to about 95% based on the weight of the coating layer.

The films may also contain additional adjuvants for coating processing such as plasticizers, polishing agents, colorants, pigments, antifoam agents, opacifiers, antisticking agents, and the like.

Various plasticizers include but are not limited to castor oil, diacetylated monoglycerides, dibutyl sebacate, diethyl phthalate, glycerin, polyethylene glycols, propylene glycols, triacetin, and triethyl citrate. Also mixtures of plasticizers may be utilized. The type of plasticizer depends upon the type of coating agent. A plasticizer is frequently present in amounts ranging from about 0% (w/w) to 30 (w/w) based on the total weight of the film coating.

An opacifier, such as titanium dioxide, can also be present in an amount ranging from about 0% (w/w) to about 20% (w/w) based on the total weight of the coating. When colored tablets are desired then the color is normally applied in the coating. Consequently, coloring agents and pigments may be present in the film coating. Various coloring agents include but are not limited to iron oxides, which can be red, yellow, black or blends thereof.

Antiadhesives can be used in the film coating process to avoid sticking effects during film formation and drying. Examples of useful antiadhesives include talc, fumed silica, or magnesium stearate. The antiadhesive can be present in the film coating in an amount of about 0% (w/w) to 15% (w/w) based upon the total weight of the coating.

Suitable polishing agents include polyethylene glycols of various molecular weights or mixtures thereof, talc, surfactants (e.g. glycerol monostearate and poloxamers and poloxamer 188), fatty alcohols (e.g., stearyl alcohol, cetyl alcohol, lauryl alcohol and myristyl alcohol) and waxes (e.g., carnauba wax, candelilla wax and white wax). For example, polyethylene glycols having molecular weights of 3,000-20,000 can be employed.

In addition to above coating ingredients, sometimes pre-formulated coating products such as OPADRY™ (supplied by Colorcon) can be used. Examples include Opadry® HP, Opadry® II white, Opadry® II yellow, Opadry® II orange, and Opadry® II brown and Opadry Blue 13B50579. Opadry® II white 85F18422 comprises polyvinyl alcohol, polyethylene glycol, titanium dioxide, and talc. These products require only mixing with a liquid before use.

Other conventional polymer coating systems may be employed, such as enteric coating systems. Suitable systems in include, but are not limited to, Eudragit R and S series resins, (acrylic acid copolymers-Rohm Pharma), cellulose acetate phthalate, cellulose acetate maleate, cellulose acetate succinate, hydroxypropylmethyl cellulose phthalate, hydroxypropylmethylcellulose acetate succinate, and the like, and a suitable plasticizer such as triethyl citrate, diethyl phthalate, tributyl citrate, triacetin, dibutyl phthalate, dibutyl sebicate, Myvacet 940, and other commonly used plasticizers as may be suitable for particular enteric polymers can be used. It will be appreciated that any polymer with suitable plasticizer can be used in aqueous or non-aqueous system to form an enteric coating

Various solvents used in the processes of preparing pharmaceutical formulations of the present invention include but are not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethyl sulphoxide, dimethyl formamide, tetrahydrofuran, and mixtures thereof.

Tablets of various sizes can be prepared in accordance with the invention. Tablets can range from about 1 mg to about 2000 mg in total weight, containing the active compounds in the ranges described above, with the remainder being a physiologically acceptable carrier of other materials according to accepted pharmaceutical practice. These tablets can also be scored to provide for fractional doses in some cases. Gelatin capsules can be similarly formulated.

In addition to solid oral dosage forms, the excipients discussed above can be used in any oral dosage form, including those dosage forms described in more detail below.

In one embodiment the composition is in a soft gelatin capsule dosage form. In this embodiment, the active ingredients can be mixed with water or an oil medium, for example peanut oil, liquid paraffin or olive oil.

Aqueous suspensions can also be provided in accordance with embodiments of the invention. An aqueous suspension can contain the active ingredients in admixture with excipients suitable for the manufacture of aqueous suspensions. Such excipients include, but are not limited to, suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable liquid, including an aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di- glycerides. In addition, fatty acids such as oleic acid find use in the preparation of injectables.

Compositions of the invention may also be in the form of a dry powder in a sterile vial, for later reconstitution and use as an injectable formulation. The dry powder can contain the active ingredients in a dry powder form. Shortly before or at the time of administration, the dry powder can be mixed with pharmaceutically acceptable excipients such as diluents, chelators, dissolution agents, solubilizing agents and stabilizers. Suitable diluents include but are not limited to diluent containing a pharmaceutically acceptable carrier, such as water. Suitable dissolution agents include but are not limited to citric acid, glutamic, succinic, aspartic, maleic, fumaric, and adipic acid, Suitable chelators include but are not limited to, ethylenediaminetetraacetic acid (EDTA), ethylene-bis(oxyethylene nitro) tetraacetic acid (EGTA), dimercaptosuccinic acid (DMSA), and CDTA (1,2-diaminocyclohexanetetraacetic acid). Suitable solubilizing agents include but are not limited to wetting agents such as polysorbates, glycerin and poloxamers, non-ionic and ionic surfactants, food acids and bases (e.g. sodium bicarbonate), and alcohols, and buffer salts for pH control. Suitable stabilizers include but are not limited to polysaccharides, such as cellulose and cellulose derivatives, and simple alcohols, such as glycerol; bacteriostatic agents such as phenol, m-cresol and methylparaben; isotonic agents, such as sodium chloride, glycerol, and glucose; lecithins, such as example natural lecithins (e.g. egg yolk lecithin or soya bean lecithin) and synthetic or semisynthetic lecithins (e.g. dimyristoylphosphatidylcholine, dipalmitoylphosphatidylcholine or distearoyl-phosphatidylcholine; phosphatidic acids; phosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelins. Compositions of the invention may also be in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Examples of such materials are cocoa butter and polyethylene glycols.

For topical use, creams, ointments, jellies, fast melt tablets, solutions or suspensions are suitable as are nebulized forms for pulmonary delivery. Topical application includes the use of mouth washes and gargles.

In one embodiment, the compositions, such as a single composition that includes harmine and insulin, can be prepared as an injectable dosage form. The formulation can be administered via a pump, pen device, such as a prefilled KwikPen device, syringe or any other typical administration vehicle for an injectable formulation known in the art. The formulations can be provided, for example, in a prefilled pen or in a vial for use with a syringe. Administration of a composition comprising both harmine and insulin does not preclude further administrations of insulin alone without harmine as separate maintenance doses, depending on a patient's fluctuation in insulin levels.

In one embodiment, the formulations can be prepared in an immediate release dosage form. An immediate release formulation in accordance with the invention refers to a pharmaceutical formulation or component thereof which releases, or delivers, one or more pharmaceutical agents substantially immediately upon administration. With respect to oral dosage forms, the delivery will result in substantially complete dissolution within about one hour (or less). The half-life of harmine is 1-3 hours. Accordingly, in one aspect of the invention, multiple doses can be administered throughout the day in order to keep the plasma concentration of harmine at a more constant level. In one aspect, the compositions of the invention are provided in an immediate release tablet form. In forming a tablet in accordance with one embodiment of the invention, the tablet may be prepared by conventional wet granulation or dry granulation (compaction) techniques. In another aspect, compositions of the invention are provided in an injectable dosage form.

In another embodiment, the formulations can be prepared in a controlled release form. Controlled release oral formulations are known. Their purpose is to modify the rate of drug release, for example to produce a constant rate of release of a drug into the gastrointestinal tract of a patient, or to delay the release of a drug into the gastrointestinal tract of a patient. See “Sustained Release Drug Delivery Systems,” pp. 3-6, edited by J R Robinson, published by Marcel Dekker Inc.). A further advantage of controlled release formulations is that side effects may be reduced due to the avoidance of initial high plasma concentrations. By administering the formulations of the invention in a controlled release manner, the dose and the toxicity of the active compounds, and specifically harmine, can be kept at lower levels while the plasma concentration of the compounds can be held to a more constant level than is experienced with an immediate release formulation.

In certain aspects, the active compounds in the compositions of the invention can be released over a period of from about 2 hours to about 24 hours, such as for about a 4, 6, 8, 10, 12, 16, 18, 20, or 24 hour period.

Controlled-release oral dosage forms release their active ingredient into the gastro-intestinal tract of a patient over a sustained period of time following administration of the dosage form to the patient. Examples of controlled-release oral dosage forms in accordance with the invention include those in which the active ingredient is embedded in a matrix from which it is released by diffusion or erosion, those in which the active ingredient is present in a core which is coated with a release rate-controlling membrane, those in which the active ingredient is present in a core provided with an outer coating impermeable to the active ingredient, the outer coating having an aperture (which may be drilled) for release of the active ingredient, those in which the active ingredient is released through a semi-permeable membrane, allowing the drug to diffuse across the membrane or through liquid filled pores within the membrane, those in which the active ingredient is present as an ion exchange complex, and those in which the active ingredient is subjected to osmotic potential triggered release, among others.

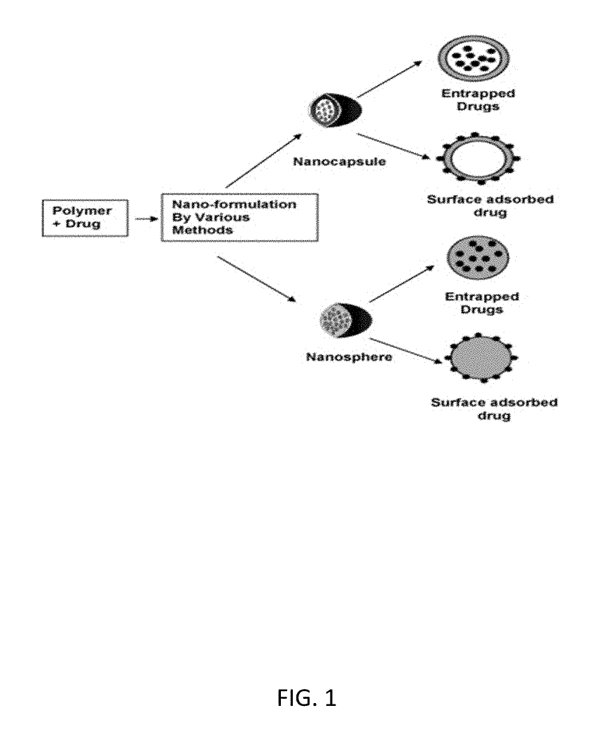

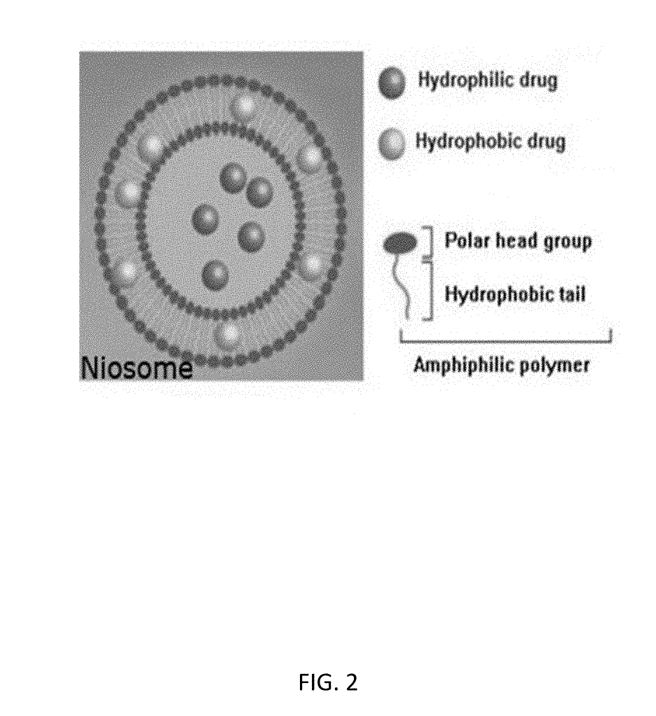

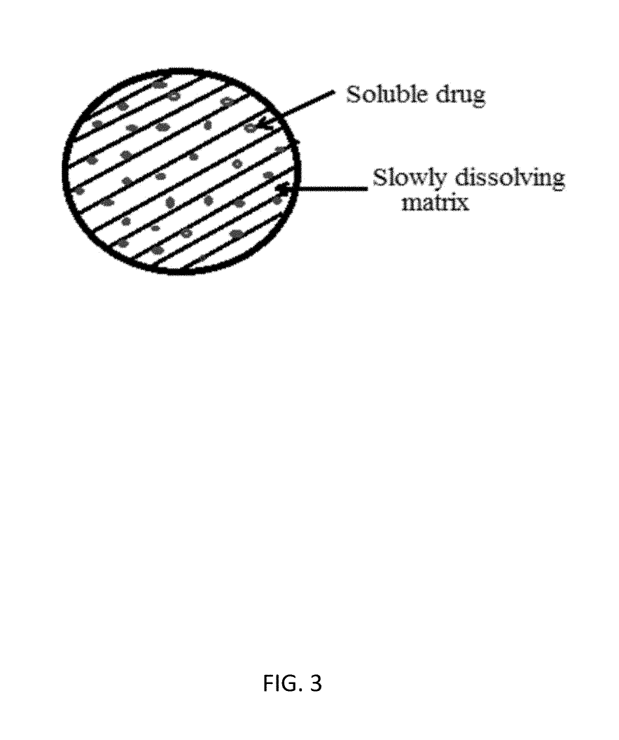

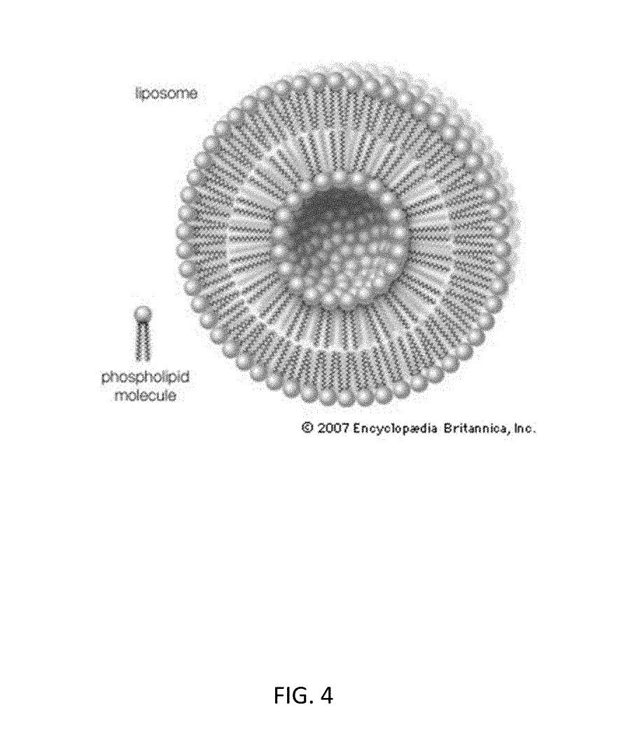

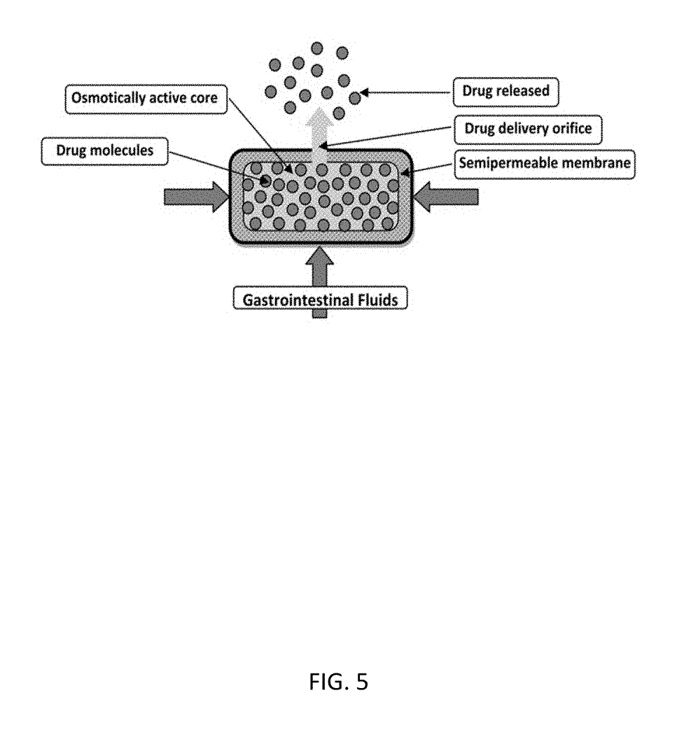

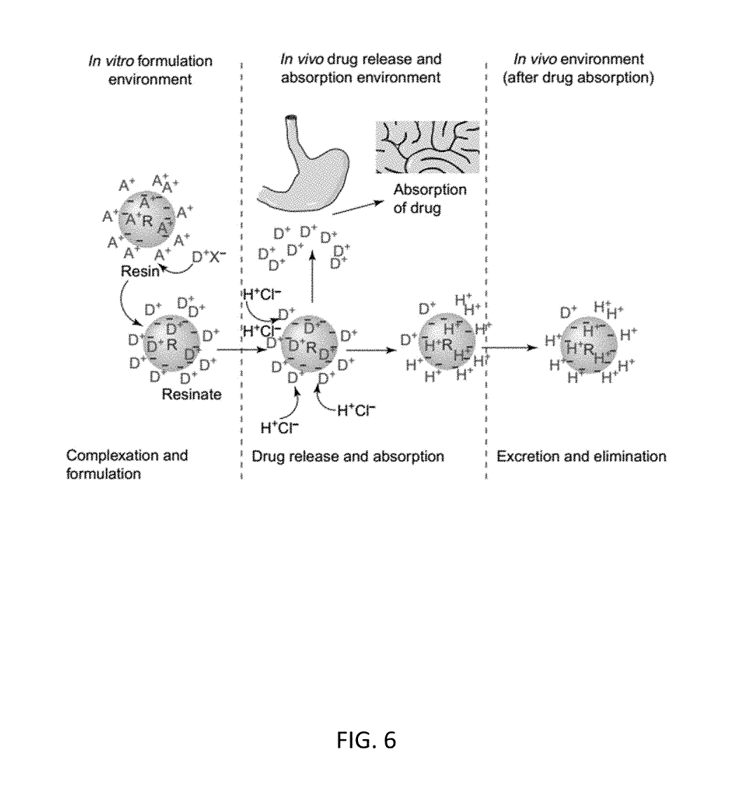

According to one aspect of the invention, the controlled release dosage form can include the drugs formulated as coated nanoparticles, as shown in FIG. 1. According to this dosage form, the nanoparticles can be coated by a polymer to form a membrane or matrix. Drug nanoparticles are released by controlled diffusion or erosion from the nanoparticle core across the polymeric membrane or matrix. The polymeric membrane coating acts as a barrier to release. Accordingly, the solubility and diffusivity of the drug in the polymer membrane becomes the determining factor in the release of the drug. Nanoparticulate delivery systems have the potential to improve drug stability, increase the duration of the therapeutic effect and permit administration through non-parental routes.

In one example of this type of composition, the controlled release of insulin resulted in a sustained hypoglycemic effect of over 24 hours. In this example, multilayered nanoparticles were formed by alginate and dextran sulphate nucleating around calcium and binding to poloxamer, which was stabilised by chitosan and coated with albumin.

In another example of this type of composition, hypoglycaemia was able to be sustained for a period of 18 hours. See B. Sarmento et al., (2007) “Alginate/Chitosan Nanoparticles are Effective for Oral Insulin Delivery”, Pharmaceutical Research, 24 (12), 2198-2206. In this example, nanoparticles were prepared by ionotropic pre-gelation of an alginate core followed by chitosan polyelectrolyte complexation. In one aspect, nanoparticles can be negatively charged and have a mean size of 750 nm. See Id. Due to the nanosize range and mucoadhesive properties, the particles are suitable for uptake within the gastrointestinal tract.

According to another aspect of the invention, the controlled release dosage form can include the active compounds formulated as nisomes, as shown in FIG. 2, which are non-ionic surfactant-based vesicles formed using non-ionic surfactant and cholesterol incorporation as an excipient. Nisomes are capable of encapsulating both hydrophilic and hydrophobic drug substances. Nisomes also have high penetration efficiency though biological membranes, and are biodegradable, relatively non-toxic, stable and inexpensive.

In one example of this embodiment using metformin HCl, a biphasic, oral release of the drug was provided over a 24 hour period. See Hasan, A., et. al., (2013) “Formulation and evaluation of metformin hydrochloride-loaded nisomes as controlled release drug delivery system” Drug Delivery, 20(3-4): 120-126.

According to another aspect of the invention, the controlled release dosage form can include the microencapsulation of the drugs, as shown in FIG. 3. In this system, the drug is coated to a desired thickness. Once in the body, the coating is slowly dissolved, thus releasing the drug contents into the gastrointestinal tract. In one aspect, the drug and rate-controlling coats can be formulated in alternating layers, to form a matrix.

In one example, a tablet formulation was provided comprising a matrix of glipizide and a combination of the hydrophobic (Eudragit RS 100) and hydrophilic (Xanthan gum) polymers in a 1:1 ratio. Oral release of glipizide was provided over a 12 hour period. See Radhika, P. R., Pal, T. K. & Sivakumar, T., 2009. “Formulation and evaluation of sustained release matrix tablets of glipizide”. Iranian Journal of Pharmaceutical Sciences, 5(4), pp. 205-214.

In yet another embodiment, compositions of the invention can be formulated as liposomes. See FIG. 4. Liposomes are composed of phospholipids whereby an aqueous core is surrounded by a lipid bilayer separating the inner aqueous core from the bulk outside. Liposomes can be coated with an outer polymer layer to promote stabilisation and to prolong their residence time in gastrointestinal tract.

One example of this embodiment includes DepoFoam technology, which consists of multivesicular liposomes characterized by their unique structure of multiple non-concentric aqueous chambers surrounded by a network of lipid membranes. By formulating insulin and peptides in a liposomal carrier, a sustained biological effect of over 1 month was achieved. See Ye, Q., et. al. (2000) “DepoFoam™ technology: a vehicle for controlled delivery of protein and peptide drugs”, Journal of Controlled Release, 64 (1-3), 155-166.

In another embodiment, compositions of the invention can be formulated as an osmotically controlled drug delivery system, as shown in FIG. 5. This system is a controlled release oral drug delivery system in the form of a tablet, wherein the tablet has a rigid water-permeable jacket with one or more small holes. As the tablet passes through the body, the osmotic pressure of water entering the tablet pushes the active drug through the opening in the tablet.

In one example of this embodiment, an oral osmotic drug delivery system for the drug nateglinide provided an oral release profile of over 12 hours. See Karri, S. R., Radhakrishna, K. & Ganesh, G. N. K., 2014. “Development of osmotically controlled oral drug delivey system for nateglinide an anti-diabetic drug,”International Journal of Pharmacy and Pharmaceutical Sciences, 6(7).

In yet another embodiment, the compositions of the invention can be formulated in an ion exchange resin. See FIG. 6. The process of ion exchange involves the reversible interchange of ions (of like charge) between a liquid and a solid phase, involving no radical change in the structure and properties of the solid. The solid phases in the ion exchange process are referred to as ion exchange resin, and are usually polymers with integrated ionic moieties. Based on the nature of the ionic species being interchanged, based on the drug(s), the ion exchange process is known as either cation exchange or anion exchange. Accordingly, the ion exchange resin used in these processes are referred to as cation-exchange resin or anion-exchange resin, respectively. For an example of this technology, see Anand, V., Kandarapu, R. & Garg, S., 2001. “Ion-exchange resins: carrying drug delivery forward”. Drug Discovery Today, 6(17), pp.905-914.

Controlled release injectable formulations can include the following types: oil-based injectable solutions, injectable-drug suspensions, polymer-based microspheres and polymer-based in-situ formings. With oil-based solutions, lipophilic drugs are typically dissolved in vegetable oils. With suspensions, lipophilic drugs are typically provided in aqueous solvents as suspensions. With respect to polymer-based microspheres, the following mechanisms can be used to slow the release of active ingredient(s): initial release from the surface, release through the pores, diffusion through the intact polymer barrier, diffusion through a water-swollen barrier, polymer erosion, and bulk degradation.

It is also to be understood that dosage forms of the invention can be provided in dual release formulations. Dual release formulations can combine the active compound(s) in both an immediate release form and a controlled-release form in one dosage form. For example with respect to oral dosage forms, a bilayer tablet can be formed with one layer containing immediate release active ingredient and the other layer containing the active ingredient embedded in a matrix from which it is released by diffusion or erosion. Alternatively, one or more immediate release beads can be combined with one or more beads which are coated with a release rate-controlling membrane in a capsule to give a dual release formulation. Sustained release formulations in which the active compound(s) are present in a core provided with an outer coating impermeable to the active compound(s), the outer coating having an aperture (which may be drilled) for release of the active compound(s), can be coated with drug in immediate release form to give a dual release formulation. Dual release formulations can also combine drug in immediate release form with additional drug in pulsed release form. For example, a capsule containing an erodible plug could liberate drug initially and after a predetermined period of time further liberate drug in immediate- or sustained-release form. Dual release formulations can also be provided as an injectable dosage form. For example, in a composition comprising insulin, two types of insulin can be included in the liquid composition—a fast-acting insulin and a longer acting insulin. As another example, one active ingredient or some of the active ingredient can be provided in a controlled release form, such as in polymer microspheres, and another portion of the active ingredient or a second active ingredient is provided in an immediate release form.

In accordance with certain aspects of the invention, controlled and dual release formulations of the invention can provide therapeutic plasma levels of one or more of the active compounds to the human patient over a 2 to 24 hour period after administration. For example, a controlled release oral dosage form can exhibit the following in vitro dissolution profile when tested in a USP Type 2 apparatus at 75 rpms in 900 ml of a pH 7.5 phosphate buffer and 37° C.:
0-25% of one or more of the active compounds is released after 2 hours;
10-50% of one or more of the active compounds is released after 4 hours;
30-90% of one or more of the active compounds is released after 8 hours;
at least 50% of one or more of the active compounds is released after 12 hours;
at least 60% of the one or more of the active compounds after 16 hours and
at least 70% of the one or more of the active compounds is released after 20 hours.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A pharmaceutical composition comprising:
harmine in an amount insufficient to stimulate pancreatic beta cell production if administered alone in a single dose; and
a pharmaceutically-acceptable excipient.

2. The composition of claim 1, further comprising one or more compounds selected from the group consisting of insulin, metformin, and repaglinide.

3. The composition of claim 1, wherein said amount of harmine is between about 0.1 mg/kg and 10 mg/kg.

4. The composition of claim 2, wherein the insulin is present in an amount of from about 20 pmol/l to about 90 pmol/l.

5. The composition of claim 2, wherein repaglinide and/or metformin is present in an amount below a threshold dose necessary to stimulate insulin production when administered alone.

6. The composition of claim 1, wherein at least one of harmine, insulin, metformin, and repaglinide is present in a pro-drug form.

7. The composition of claim 1, wherein said harmine is conjugated to a blocking group selected from a hydroxyl group, an amine group, a carboxyl group, a carbonyl group, and an amide group.

8. The composition of claim 7, wherein said harmine is conjugated to an acetate blocking group as indicated below: 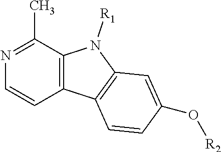
wherein R₁is C₂H₃O and R₂ is H; or R₁ is H and R₂ is C₂H₃O.

9. A pharmaceutical composition comprising:
harmine or INDY in an amount that would be insufficient to stimulate pancreatic beta cell production in a single administration to a patient when the concentration of harmine in the patient is zero.

10. The composition of claim 9, wherein said amount of harmine is less than about 1.0 mg/kg.

11. The composition of claim 9, further comprising one or more compounds selected from the group consisting of insulin or an analog thereof, repaglinide, and metformin.

12. The composition of claim 11, wherein repaglinide and/or metformin is present in an amount below a threshold dose necessary to stimulate insulin production when administered alone.

13. The composition of claim 9, wherein harmine is present in a pro-drug form.

14. The composition of claim 11, wherein at least one of said harmine, insulin, repaglinide, or metformin is present in a pro-drug form.

15. The composition of claim 9, wherein said harmine is conjugated to an acetate protecting group.

16. The composition of claim 15, wherein said harmine conjugated to an acetate blocking group comprises the formula: 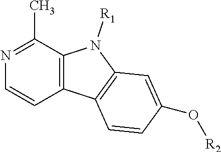
wherein R₁is C₂H₃O and R₂ is H; or R₁ is Hand R₂ is C₂H₃O.

17. The composition of claim 9, wherein said harmine is present in an amount of from about 0.1 mg/kg to 10 mg/kg.

18. The composition of claim 1, wherein said composition is provided in a controlled release dosage form.

19. The composition of claim 1, wherein said composition is formulated to be administered once a day.

20. A pharmaceutical composition comprising:
harmine in an amount insufficient to cause psychotoxic effects;
one or more compounds selected from the group consisting of insulin or an analog thereof, metformin, and repaglinide; and
a pharmaceutically-acceptable excipient.
